(19) United States
(12) Patent Application Publication
(10) Pub. No.: US 2015/0257835 A1
(43) Pub. Date: Sep. 17, 2015

(54) GLOVE WITH REMOVAL TAB

(71) Applicant: John Norman Le Blanc, Carmel Valley, CA (US)

(72) Inventor: John Norman Le Blanc, Carmel Valley, CA (US)

(21) Appl. No.: 14/644,708

(22) Filed: Mar. 11, 2015

Related U.S. Application Data
(60) Provisional application No. 61/953,492, filed on Mar. 14, 2014.

(51) Int. Cl. A61B 19/04; A41D 19/015

(52) U.S. Cl. CPC A61B 19/04; A41D 19/015; A41D 2400/52; A41D 2600/20

(57) ABSTRACT

Some embodiments of the present disclosure include a glove for preventing contamination when removed. The glove may include a glove configured to accommodate a user's hand, and a removal tab extending from an edge of the glove proximate to an opening in the glove. In embodiment, the glove may be made of any desired material, such as latex or nitrile. Furthermore, the tab may be integrated into the manufacture of the glove or may be added after the manufacture of the glove.

### RELATED APPLICATION

This application claims priority to provisional patent application U.S. Ser. No. 61/953,492 filed on Mar. 14, 2014, the entire contents of which is herein incorporated by reference.

### BACKGROUND

The embodiments herein relate generally to sterile environments, and more particularly, to a glove, such as a medical glove, with a removal tab.

Many occupations, particularly in the medical field, require the use of gloves to reduce or prevent contamination of any sort. Gloves aid in preventing cross-contamination of disease, bodily fluids, and blood pathogens. However, removing a medical glove without touching any part of the body, such as the user's wrist, or an accessory, such as a wrist watch, is very difficult.

In triage, medical personnel may quickly move from patient to patient. Oftentimes, the medical personnel will layer gloves so that only removing the top layer is required between patients. However, when gloves are layered, it is very difficult to remove the top glove without touching the glove layered below the top glove. Specifically, when removing a blood covered in fluid, such as blood, it is almost impossible to remove the glove without getting a finger under the top edge of the glove, which can compromise the safety of the medical personnel.

Therefore, what is needed is a mechanism to aid a user in removing a glove, while also helping the user prevent touching the glove being removed to any adjacent surface.

### SUMMARY

Some embodiments of the present disclosure include a glove for preventing contamination when removed. The glove may include a glove configured to accommodate a user's hand, and a removal tab extending from an edge of the glove proximate to an opening in the glove. In embodiment, the glove may be made of any desired material, such as latex or nitrile. Furthermore, the tab may be integrated into the manufacture of the glove or may be added after the manufacture of the glove.

### BRIEF DESCRIPTION OF THE FIGURES

The detailed description of some embodiments of the invention is made below with reference to the accompanying figures, wherein like numerals represent corresponding parts of the figures.

FIG. 1 is a top view of one embodiment of the present invention.

FIG. 2 is a perspective view of one embodiment of the present invention, demonstrating an initial stage of glove removal.

FIG. 3 is a perspective view o one embodiment of the present invention, demonstrating complete removal of the glove.

### DETAILED DESCRIPTION OF CERTAIN EMBODIMENTS

In the following detailed description of the invention, numerous details, examples, and embodiments of the invention are described. However, it will be clear and apparent to one skilled in the art that the invention is not limited to the embodiments set forth and that the invention can be adapted for any of several applications.

The device of the present disclosure may be used to remove a glove and may comprise the following elements. This list of possible constituent elements is intended to be exemplary only, and it is not intended that this list be used to limit the device of the present application to just these elements. Persons having ordinary skill in the art relevant to the present disclosure may understand there to be equivalent elements that may be substituted within the present disclosure without changing the essential function or operation of the device.
1. Glove
2. Tab

The various elements of the glove with a removal tab for protecting a user from contaminants existing on the outer surface of the glove of the present disclosure may be related in the following exemplary fashion. It is not intended to limit the scope or nature of the relationships between the various elements and the following examples are presented as illustrative examples only.

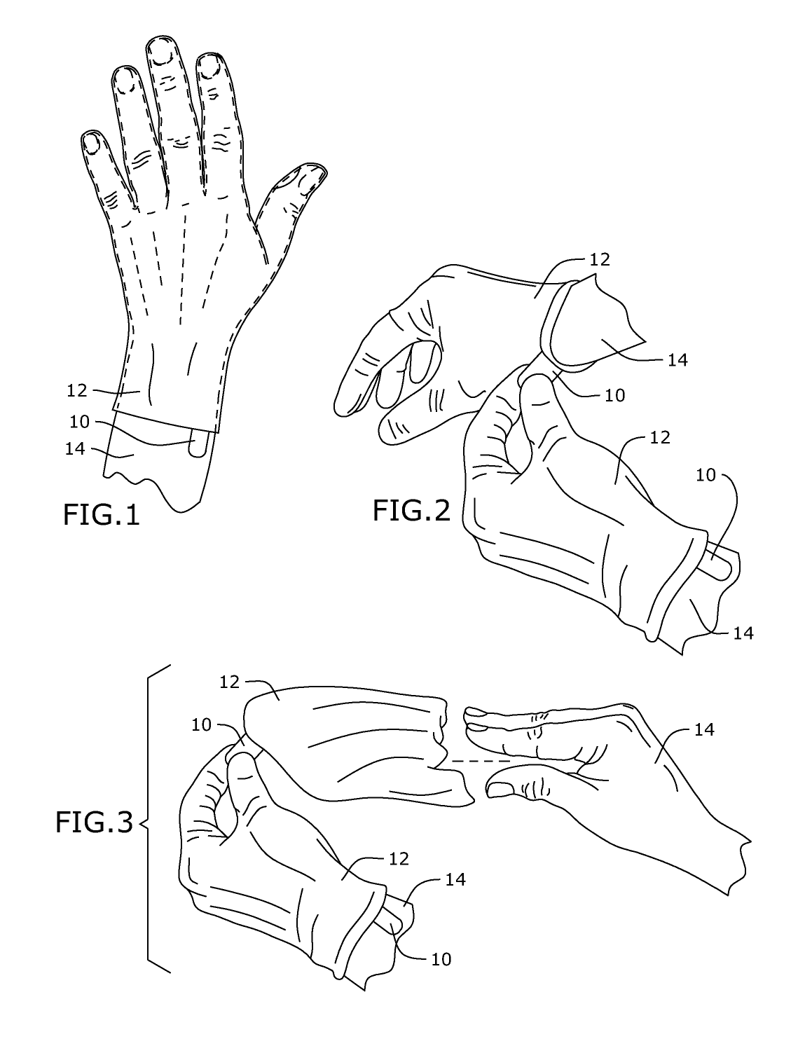

By way of example, and referring to FIGS. 1-3, some embodiments of the glove of the present disclosure comprise a glove 12 configured to accommodate a user's 14 hand and a removal tab 10 extending from an edge of the glove 12 proximate to the opening of the glove 12. For example, as shown in FIGS. 1-3, the removal tab 10 may be positioned proximate to the user's thumb.

To make the glove of the present disclosure, a glove 12 may be made as conventional with a tab 10 added to the glove 12. For example, a material used to make the glove 12 may be sprayed onto a mold, including the addition of a tab 10 proximate to the opening of the glove 12. The glove 12 may then be removed from the mold with a burst of air. In other embodiments, the tab 10 may be added to the glove 12 using any suitable method.

To use the glove of the present disclosure, a user 14 would wear the glove 12 as normal. To remove the glove 12, the user 14 may pull on the removal tab 10, which may result in the glove 12 rolling up and enveloping the outer surface into an interior of the glove. Additionally, using the removal tab 10 to remove the glove 12 may prevent the user's skin, uniform, watch, layered gloves, and the like from being contaminated by the outer surface of the glove 12.

The glove 12 of the present disclosure may be made of any desired material, such as latex, nitrile, or any other desired material. The tab 10 may be made of the same material as the glove 12 and may be an extension of the glove 12. In other embodiments, the tab 10 may comprise an alternate material and may be added to the glove 12 after the manufacture of the glove 12.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A glove for preventing contamination when removed, the glove comprising:
a glove configured to accommodate a user's hand; and
a removal tab extending from an edge of the glove proximate to an opening in the glove.

2. The glove of claim 1, wherein the removal tab is positioned on the edge of the glove closest to the user's thumb.

3. The glove of claim 1, wherein the glove and the removal tab comprise a material selected from the group consisting of latex and nitrile.

4. The glove of claim 1, wherein the glove and the removal tab comprise the same material.

5. A method for making a glove for preventing contamination when removed, the method comprising:
spraying a material onto a mold of a hand, the mold including a removal tab portion, creating a glove; and
removing the glove from the mold with a burst of air,
wherein the glove comprises:
a glove configured to accommodate a user's hand; and
a removal tab extending from an edge of the glove proximate to an opening in the glove.

6. The method of claim 5, wherein the removal tab is positioned on the edge of the glove closest to the user's thumb.

7. The method of claim 5, wherein the glove and the removal tab comprise a material selected from the group consisting of latex and nitrile.
